(19) United States
(12) Patent Application Publication
(10) Pub. No.: US 2008/0177297 A1
(43) Pub. Date: Jul. 24, 2008

(54) Forceps

(75) Inventors: Anton J. Steiner, Wharton, NJ (US); Brian J. Cole, Chicago, IL (US)

Correspondence Address: JOHN S. HALE;GIPPLE & HALE, 6665-A OLD DOMINION DRIVE, MCLEAN, VA, 22101, US

(73) Assignee: Musculoskeletal Transplant Foundation

(21) Appl. No.: 11/657,041

(22) Filed: Jan. 24, 2007

(51) Int. Cl. A61B 17/00

(52) U.S. Cl. 606/205

(57) ABSTRACT

The invention is directed toward a surgical forceps comprising a pair of scissor arms connected together by a pivot where the proximal ends forms a hand grasping surface and the distal ends are provided with jaw members. Each jaw member has a curved inner surface with a plurality of teeth and measurement markings at spaced intervals along an end surface of the first and second jaws. Each jaw member is semi-circular in shape and extends outward from each respective arm at an angle of about 110° so that when said scissor arms are closed together the jaw ends move toward each other. A rachet assembly is mounted on one scissor arm to engage a pawl mounted on the other arm to lock the jaw members in a fixed position.

### RELATED APPLICATIONS

There is no related application.

### STATEMENT REGARDING FEDERALLY SPONSORED RESEARCH OR DEVELOPMENT

Not applicable.

### REFERENCE TO SEQUENCE LISTING, A TABLE OR A COMPUTER PROGRAM LISTING COMPACT DISC APPENDIX

None.

### BACKGROUND OF THE INVENTION

1. Field of Invention

The present invention is generally directed toward the surgical treatment of articular chondral defects and is more specifically directed toward a surgical forceps for holding a cylindrical allograft cartilage implant plug having a cartilage face and bone body to allow trimming of the same.

2. Description of the Prior Art

Articular cartilage injury and degeneration present medical problems to the general population which are constantly addressed by orthopedic surgeons. Every year in the United States, over 500,000 arthroplastic or joint repair procedures are performed. These include approximately 125,000 total hip and 150,000 total knee arthroplastics and over 41,000 open arthroscopic procedures to repair cartilaginous defects of the knee.

In the knee joint, the articular cartilage tissue forms a lining which faces the joint cavity on one side and is linked to the subchondral bone plate by a narrow layer of calcified cartilage tissue on the other. Articular cartilage (hyaline cartilage) consists primarily of extracellular matrix with a sparse population of chondrocytes distributed throughout the tissue. Articular cartilage is composed of chondrocytes, type II collagen fibril meshwork, proteoglycans and water. Active chondrocytes are unique in that they have a relatively low turnover rate and are sparsely distributed within the surrounding matrix. The collagens give the tissue its form and tensile strength and the interaction of proteoglycans with water give the tissue its stiffness to compression, resilience and durability. The hyaline cartilage provides a low friction bearing surface over the bony parts of the joint. If the cartilage lining becomes worn or damaged resulting in lesions, joint movement may be painful or severely restricted. Whereas damaged bone typically can regenerate successfully, hyaline cartilage regeneration is quite limited because of it's limited regenerative and reparative abilities.

Articular cartilage lesions generally do not heal, or heal only partially under certain biological conditions due to the lack of nerves, blood vessels and a lymphatic system. The limited reparative capabilities of hyaline cartilage usually results in the generation of repair tissue that lacks the structure and biomechanical properties of normal cartilage. Generally, the healing of the defect results in a fibrocartilaginous repair tissue that lacks the structure and biomedical properties of hyaline cartilage and degrades over the course of time. Articular cartilage lesions are frequently associated with disability and with symptoms such as joint pain, locking phenomena and reduced or disturbed function. These lesions are difficult to treat because of the distinctive structure and function of hyaline cartilage. Such lesions are believed to progress to severe forms of osteoarthritis. Osteoarthritis is the leading cause of disability and impairment in middle-aged and older individuals, entailing significant economic, social and psychological costs. Each year, osteoarthritis accounts for as many as 39 million physician visits and more than 500,000 hospitalizations. By the year 2020, arthritis is expected to affect almost 60 million persons in the United States and to limit the activity of 11.6 million persons.

There are many current therapeutic methods being used. None of these therapies has resulted in the successful regeneration of hyaline-like tissue that withstands normal joint loading and activity over prolonged periods. Currently, the techniques most widely utilized clinically for cartilage defects and degeneration are not articular cartilage substitution procedures, but rather lavage, arthroscopic debridement, and repair stimulation. The direct transplantation of cells or tissue into a defect and the replacement of the defect with biologic or synthetic substitutions presently accounts for only a small percentage of surgical interventions. The optimum surgical goal is to replace the defects with cartilage-like substitutes so as to provide pain relief, reduce effusions and inflammation, restore function, reduce disability and postpone or alleviate the need for prosthetic replacement.

Lavage and arthroscopic debridement involve irrigation of the joint with solutions of sodium chloride, Ringer or Ringer and lactate. The temporary pain relief is believed to result from removing degenerative cartilage debris, proteolytic enzymes and inflammatory mediators. These techniques provide temporary pain relief, but have little or no potential for further healing.

Repair stimulation is conducted by means of drilling, abrasion arthroplasty or microfracture. Penetration into the subchondral bone induces bleeding and fibrin clot formation which promotes initial repair, however, the tissue formed is fibrous in nature and not durable. Pain relief is temporary as the tissue exhibits degeneration, loss of resilience, stiffness and wear characteristics over time.

The periosteum and perichondrium have been shown to contain mesenchymal progenitor cells capable of differentiation and proliferation. They have been used as grafts in both animal and human models to repair articular defects. Few patients over 40 years of age obtained good clinical results, which most likely reflects the decreasing population of osteochondral progenitor cells with increasing age. There have also been problems with adhesion and stability of the grafts, which result in their displacement or loss from the repair site.

Osteochondral transplantation or mosaicplasty involves excising all injured or unstable tissue from the articular defect and creating cylindrical holes in the base of the defect and underlying bone. These holes are filled with autologous cylindrical plugs of healthy cartilage and bone in a mosaic fashion. The osteochondral plugs are harvested from a lower weight-bearing area of lesser importance in the same joint. Reports of results of osteochondral plug autografts in a small numbers of patients indicate that they decrease pain and improve joint function, however, long-term results have not been reported. Factors that can compromise the results include donor site morbidity, effects of joint incongruity on the opposing surface of the donor site, damage to the chondrocytes at the articular margins of the donor and recipient sites during preparation and implantation, and collapse or settling of the graft over time. The limited availability of sites for harvest of osteochondral autografts restricts the use of this approach to treatment of relatively small articular defects and the healing of the chondral portion of the autograft to the adjacent articular cartilage remains a concern.

Transplantation of large allografts of bone and overlying articular cartilage is another treatment option that involves a greater area than is suitable for autologous cylindrical plugs, as well as for a non-contained defect. The advantages of osteochondral allografts are the potential to restore the anatomic contour of the joint, lack of morbidity related to graft harvesting, greater availability than autografts and the ability to prepare allografts in any size to reconstruct large defects. Clinical experience with fresh and frozen osteochondral allografts shows that these grafts can decrease joint pain, and that the osseous portion of an allograft can heal to the host bone and the chondral portion can function as an articular surface. Drawbacks associated with this methodology in the clinical situation include the scarcity of fresh donor material and problems connected with the handling and storage of frozen tissue. Fresh allografts carry the risk of immune response or disease transmission. Musculoskeletal Transplant Foundation (MTF) has preserved fresh allografts in a media that maintains a cell viability of 50% for 35 days for use as implants. Frozen allografts lack cell viability and have shown a decreased amount of proteoglycan content which contribute to deterioration of the tissue.

Various studies have also been undertaken by Musculoskeletal Transplant Foundation to utilize allograft cylindrical cartilage capped bone plugs for cartilage defect replacement. When using allograft plug replacement the cylindrical plug is handled by the surgeon so that it can be trimmed to a correct length for the specific application. Consequently a need for an improved cylindrical bone plug handling forceps is necessary to allow the surgeon to easily handle the cylindrical plug during the plug trimming and implantation process. A number of patents have been directed toward clamps or forceps for holding allograft and autograft cylindrical plugs so that the same can be trimmed to size to fit into the cut bore of the excised area or to hold the cylindrical plug for insertion into the cut bore.

U.S. Pat. Nos. 6,488,033 and 6,852,114 (a divisional application of the '033 patent) issued respectively Dec. 3, 2002 and Feb. 8, 2005 are directed toward an osteochondral transplant workstation for cutting a core out of an allograft bone held in an adjustable vise with a lubricated rotary cutting bit. The core is removed from the bit, held in a specially designed set of pliers, and cut to size by a saw blade to fit into a blind bore which has been drilled into the patient's arthritic defect area.

Bone clamps or pliers are well known in the medical profession for various uses. Bone clamps are reusable devices and therefore longevity is a desirable characteristic. Generally, bone clamps are utilized to move broken bones into aligned position or hold bone fragments together while surgical procedures (e.g., installation of a screw, plate, pin, or wire) are performed. When performing surgery to repair a broken bone, it is important to clamp the bone fragments together while a mending device (e.g., a screw, plate, pin, or wire) is being installed so that the bone fragments can be maintained in alignment with substantially no gaps therebetween. For example, bone clamps may be utilized to hold bone plates in position across a bone fracture and/or to align the fractured bones while the bone plate(s) are affixed thereto or to place bone plugs in B-T-B surgery.

Typically, bone clamps utilize a rachet mechanism to control movement of the bone clamp and to maintain the bone clamp in locked position once it is operatively positioned. Ratchet mechanisms utilized with prior art bone clamps are generally of two types: (1) a unidirectional rachet, e.g., of the type utilized with standard forceps, and (2) a bidirectional ratchet having a selectively actuatable lock mechanism to retain the pawl in locked position between two consecutive rachet teeth.

U.S. Pat. No. 5,697,933 issued Dec. 16, 1997 is directed toward a bone-tendon-bone drill guide with a pair of scissor arms connected at a pivot with jaws at one end which include curved surfaces for engaging a bone end and a straight ratchet brace that is pivotally connected to the lower end of one scissoring arm. The jaws are provided with marking indicia. The straight brace pivots an edge into alignment with a single tooth that extends from the bottom end of the other scissoring arm, the straight brace including a series of teeth formed along its edge to engage the single tooth and is spring biased to urge the series of teeth against the single tooth.

U.S. Pat. No. 6,159,217 issued Dec. 12, 2000 discloses a trochlear clamp having curved jaws with the internal surfaces provided with a plurality of teeth, one of the arms being provided with a rachet assembly to hold the arms and jaws in a fixed position.

U.S. Pat. No. 5,578,032 issued Nov. 26, 1996 discloses a bone clamp with a rachet mechanism formed on the proximal ends of pivotable scissor arms and a caliper type clamp mounted on the distal ends of the scissor arms.

U.S. Pat. No. 6,315,780 issued Nov. 13, 2001 is directed toward a bone clamp for dynamic and non dynamic compression of transverse fractures with toothed jaw clamps located at the distal ends of pivotable scissor arms and a rachet mechanism located at the proximal ends of the scissor arms.

It is desirable to have a forceps instrument for properly positioning a cutting guide to ensure the accuracy in the trimming of an osteochondral bone core.

The present invention was designed to overcome prior art instruments and provide a simple to use core preparation devise which accurately fits into the patient's bore area to form a uniform cartilage surface for the patient.

### SUMMARY OF THE INVENTION

A forceps for the preparation of osteochondral allograft cartilage implants having a pivotable scissor arms with distal curved jaws to hold implant replacement cores. The curved jaws have a plurality of inner teeth to hold the core implant for trimming and are locked in position with a spring loaded rachet mechanism and pawl located on respective scissor arms.

It is an object of the invention to provide a surgical forceps for forming osteochondral allograft plugs with a cartilage layer which can be locked to provide jaw members which are fixed in position.

It is also an object of the invention to provide a surgical forceps allowing easy grasping of a cartilage repair implant which has a cartilage layer contoured to the defect site;

It is further an object of the invention to provide a surgical forceps which can be easily used by the surgeon to create correctly dimensional and contoured cartilage implants.

It is yet another object of the invention to provide a surgical forceps which can be easily cleaned and sterilized.

It is still another object of the invention to provide forceps with marking indicia along the jaw members so that accurate core lengths for the implant can be obtained.

These and other objects, advantages, and novel features of the present invention will become apparent when considered with the teachings contained in the detailed disclosure along with the accompanying drawings.

### BRIEF DESCRIPTION OF THE DRAWINGS

FIG. 1 is a perspective view of the forceps invention with the clamp jaws in position;

FIG. 2 is a perspective view of the forceps invention of FIG. 1 with the clamp jaws in a closed position holding a cartilage plug workpiece which is shown in phantom;

FIG. 3 is a top plan view of the forceps of FIG. 2;

FIG. 4 is a bottom plan view of the forceps of FIG. 2;

FIG. 5 is a rear elevation view of the forceps shown in FIG. 2;

FIG. 6 is a partial enlarged side elevation view of the forceps arm taken from the pawl arm side;

FIG. 7 is an enlarged perspective view of the clamp jaws of the forceps;

FIG. 8 is an enlarged top plan view of the clamp jaws shown in FIG. 7;

FIG. 9 is a partial enlarged view of the jaw teeth shown in Circle A of FIG. 8;

FIG. 10 is a front elevation view of the clamp jaws shown in FIG. 8; and

FIG. 11 is a side elevation view of the clamp jaws shown in FIG. 8.

### DESCRIPTION OF THE INVENTION

The term “tissue” is used in the general sense herein to mean any transplantable or implantable tissue such as bone.

The terms “transplant” and “implant” are used interchangably to refer to tissue (xenogeneic or allogeneic) which may be introduced into the body of a patient to replace or supplement the structure or function of the endogenous tissue.

The terms “autologous” and “autograft” refer to tissue or cells which originate with or are derived from the recipient, whereas the terms “allogeneic” and “allograft” refer to tissue which originate with or are derived from a donor of the same species as the recipient. The terms “xenogeneic” and “xenograft” refer to tissue which originates with or are derived from a species other than that of the recipient.

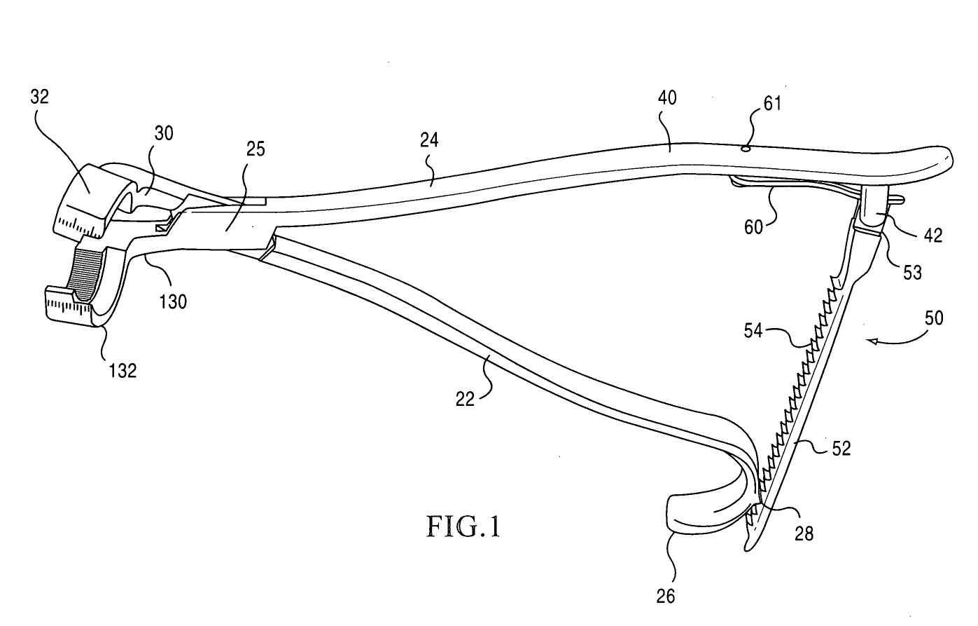

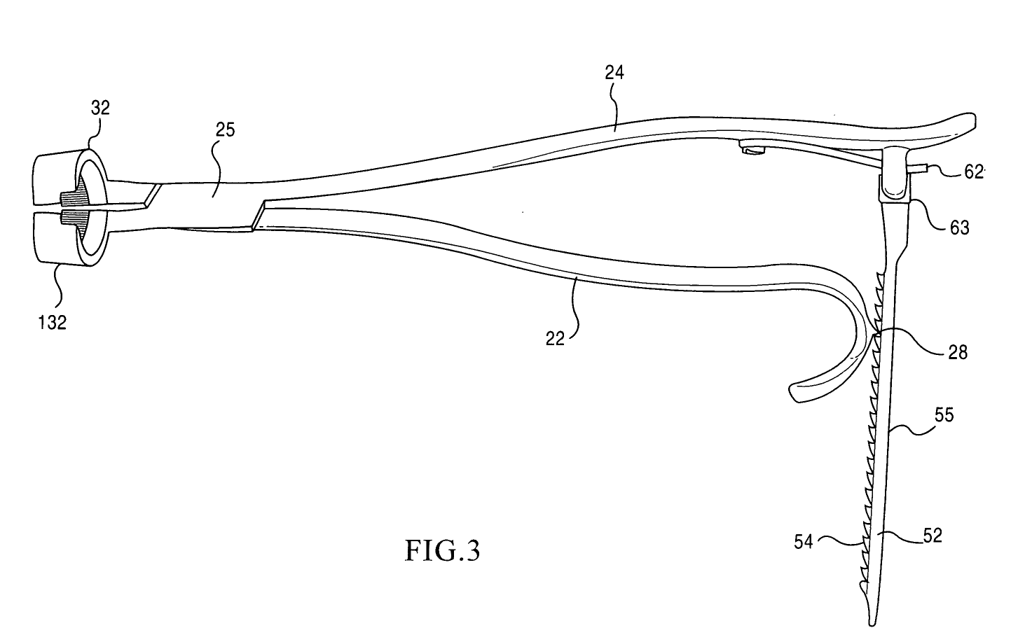

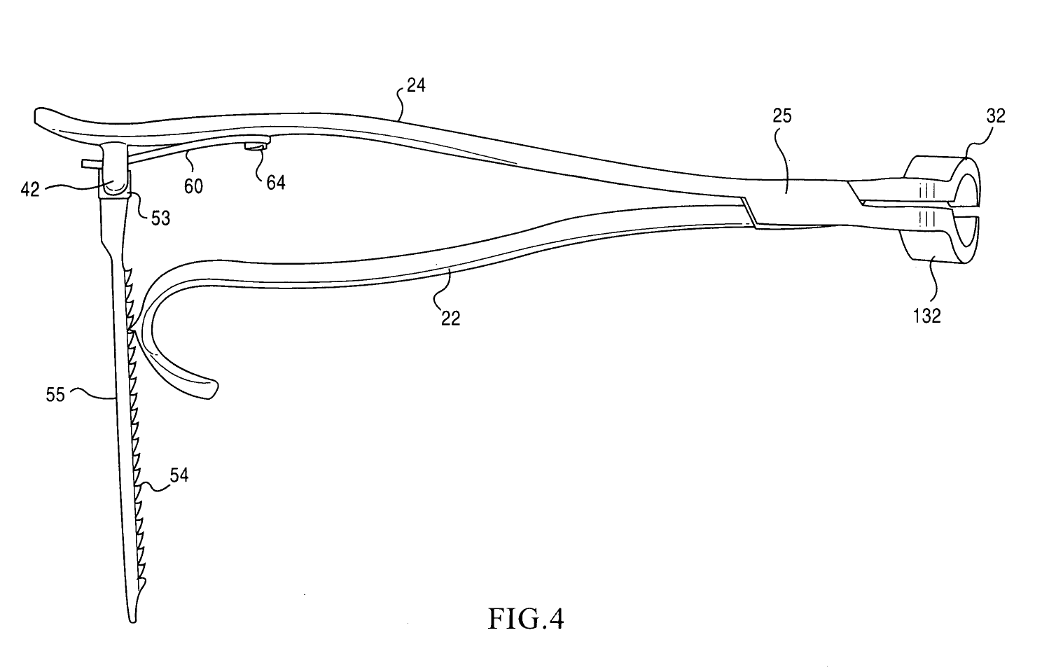

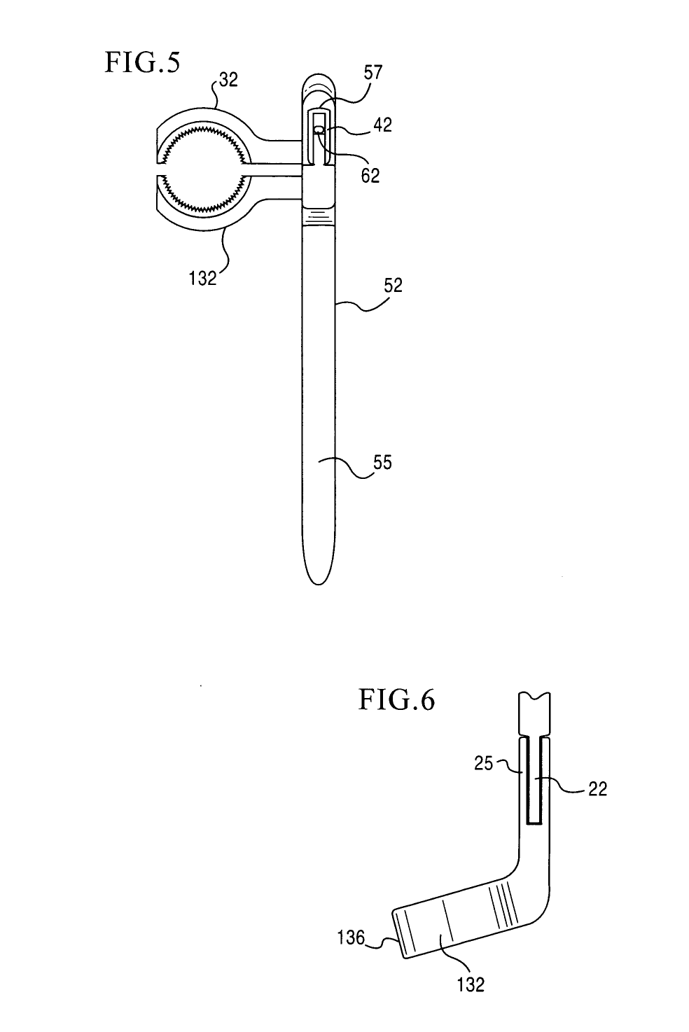

The present invention is directed towards a implant holding forceps 20 preferably constructed of 410 or 420 stainless steel. The preferred embodiment and best mode of the invention is shown in FIGS. 1-11. In the inventive forceps 20, a workpiece in the form of an allograft plug or core 200 having a cartilage cap 202 and a bone base 204 which is held in the forceps 20 for trimming for implantation into a patient.

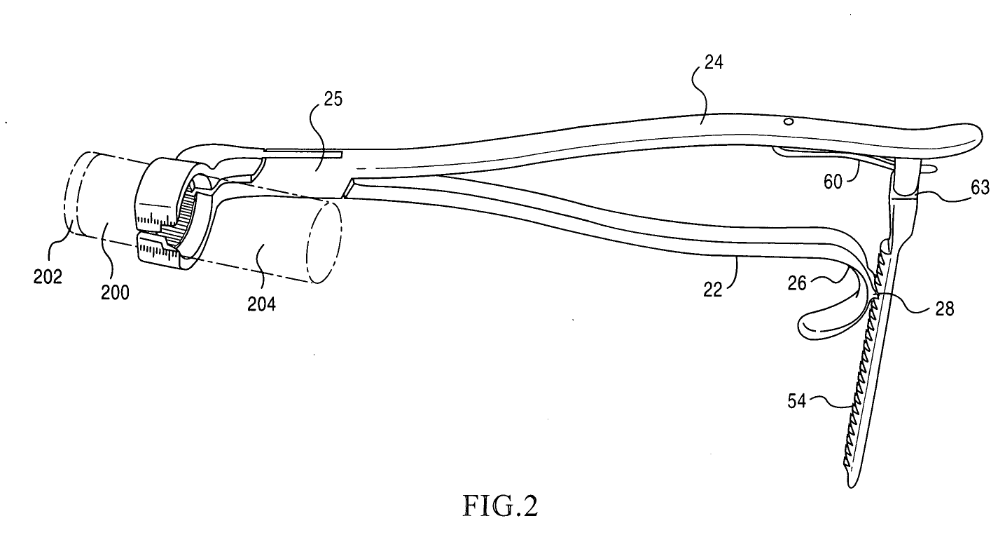

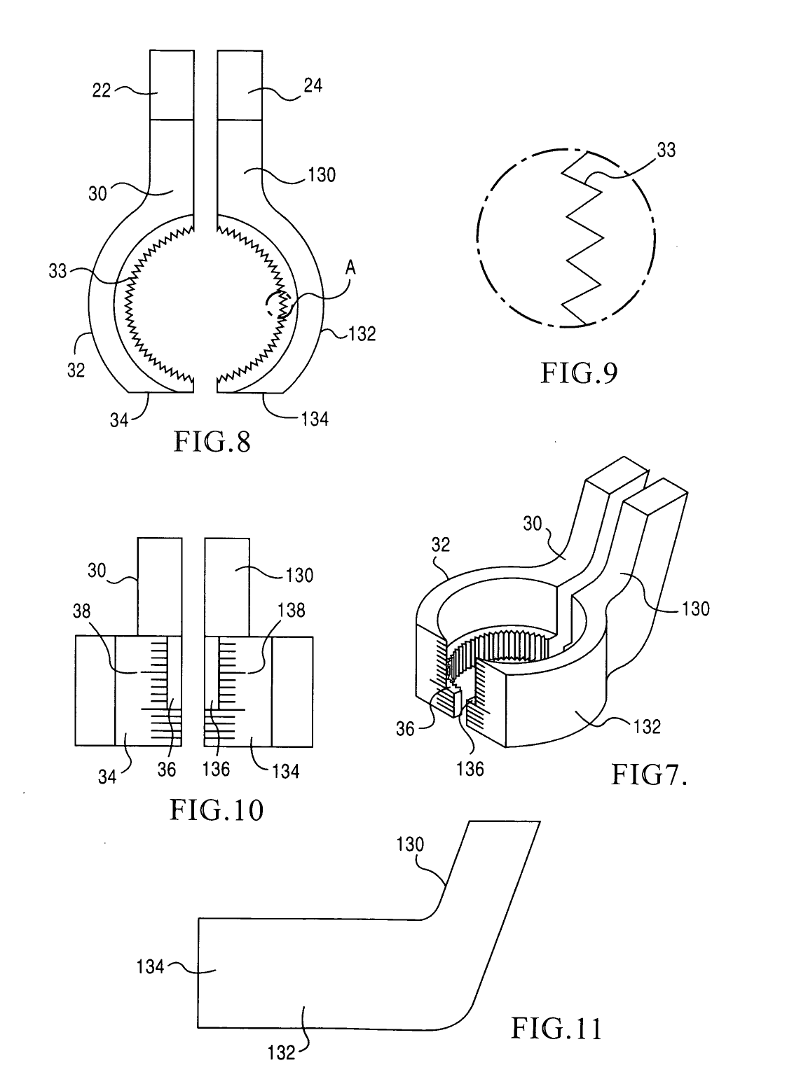

The forceps 20 has a pair of scissor arms 22 and 24 which are pivotally connected together by a pivot, (not shown) located in a pivot housing 25 as shown in FIGS. 1 and 2 which is formed by arm 24. The proximal end of arm 22 is formed with an inwardly curved grasping end 26 with the external surface of the curved end 26 being provided with a tooth or pawl 28 located at about the mid point of the curved end 26. The distal end of arm 22 has an angled neck portion 30 with a curved jaw member 32 extending therefrom. The angled neck portion 30 is angled in a range of 90° to 120° preferably 110° from the plane of the pivot housing 25. The curved jaw member 32 which is in the form of a semi-circle has a plurality of teeth 33 located around its inner curved surface extending from the distal end of the jaw up to the base of notched cutout 36, each tooth preferably forming a 60° angle with an adjacent tooth. The distal end 34 of jaw member 32 is preferably planar and is formed with a notched cutout 36 with the planar surface being provided with laser cut measurement indicia 38 having a 0.2 line thickness. The spaced measurement indicia 38 are preferably in millimeters and are scribed at spaced intervals along the end surface of each jaw as shown in detail in FIGS. 7 and 10 so that the implant core 200 can be trimmed to an exact length for placement in a bore cut in the patient when the cartilage defect is removed. The markings 38 determine locations along the implant core length for trimming the core to a length which fit into the patients bore formed by removing the defect area.

The proximal end of arm 24 is ergonomically curved at 40 and has a pivoting rachet assembly 50 mounted on the distal end. The rachet assembly 50 comprises a yoke 42 extending outward from the inner surface of the arm 24 holding the pivot base section 53 of a straight rachet bar body 52. The pivot base section 53 is stepped from the rachet bar body and has planar side surfaces which are pivotally mounted to the yoke 42 by pin means not shown. A plurality of one directional teeth 54 are formed on the inner surface of rachet bar body 52 and the outer surface 55 of the rachet bar body is smooth and planar. The teeth 54 engage a tooth or pawl 28 extending from arm 22 allowing the arms 22 and 24 and their respective jaws to be held in a fixed position with respect to each other. The end 57 of pivot section 53 of the bar body is engaged by a steel leaf spring 60 having a tip 62 which extends through yoke 42, the spring being mounted to arm 24 by a screw or fastener 64 which extends through a hole in the spring 60 and through a threaded hole 61 formed in arm 24. Thus the rachet bar body 52 is urged inwardly towards the jaws by spring 60 with arm 22 and pawl 28 driving the bar body 52 back against the spring bias. The spring biasing is overcome by lifting the bar body away from the single tooth 28

The distal end of arm 24 has an angled neck portion 130 and a curved jaw member 132. The angled neck portion 130 is angled in a range of 90° to 120° from the plane of the top surface of the jaw member 132. The curved jaw member 132 has a plurality of teeth 33 around a portion of its inner curved surface, each tooth preferably forming a 60° angle with the adjacent tooth and is positioned identical to that of the opposing jaw 32. The end 134 of jaw member 132 is preferably planar and has a notched cutout 136 with the planar surface being provided with measurement indicia 138, which is the same as measurement indicia 38.

The principles, preferred embodiments and modes of operation of the present invention have been described in the foregoing specification. However, the invention should not be construed as limited to the particular embodiment which have been described above. Variations and changes may be made by others without departing from the scope of the present invention as defined by the following claims:

1. A surgical forceps comprising a pair of scissor arms connected together by a pivot with the proximal ends of each arm forming hand grasping means and the distal ends of each arm provided with jaw means, said jaw means being angularly offset from a plane taken along a top surface of said arms, each jaw means having an opposed curved inner surface with a plurality of teeth and measurement markings placed at spaced intervals along opposing surfaces of the first and second jaw means.

2. A surgical forceps as claimed in claim 1 wherein said jaw means each comprise a semi-circular member so that when said scissor arms are closed together the jaw ends are positioned adjacent each other, each jaw member defining a cutout notch area allowing visual identification of a bone workpiece held therein.

3. A surgical forceps as claimed in claim 2 wherein said semi-circular members extend from each respective arm at an angle ranging from 90° to 130°.

4. A surgical forceps as claimed in claim 3 wherein said angle is about 110°.

5. A surgical forceps as claimed in claim 1 wherein said teeth means comprises a plurality of spaced teeth positioned along an inner curved section.

6. A surgical forceps as claimed in claim 5 wherein said plurality of spaced teeth form an angle of about 60° with respect to each other.

7. A surgical forceps as claimed in claim 1 wherein one of said scissor arms has an outwardly curved proximal end with a pawl formed on an outside surface of said curved end and the other scissor arm is provided with a ratchet member assembly on its proximal end which is adapted to engage said pawl to hold the arms of said forceps in a fixed position.

8. A surgical forceps as claimed in claim 7 wherein said ratchet member assembly comprises a linear bar with a plurality of identically shaped unidirectional teeth, said linear bar being pivotally mounted on said other arm, said linear bar being engaged and urged toward said jaw means by a spring member mounted on said other arm.

9. A surgical forceps comprising a pair of scissor arms connected together by a pivot with the proximal ends of each arm forming hand grasping means and the distal ends of each arm being provided with jaw means, said jaw means each comprising a semi-circular member extending outward from each respective arm at an angle ranging from 90° to 130° so that when said scissor arms are closed together the jaw ends move toward each other, each semi-circular member defining a cutout notch area allowing visual identification of a workpiece held therein, and defining a curved inner surface with teeth formed on said curved inner surface and measurement markings placed at spaced intervals along an end surface of the first and second semi-circular members.

10. A surgical forceps as claimed in claim 9 wherein said teeth are equally spaced and are positioned at the distal end of each jaw member below the cutout notch area.

11. A surgical forceps as claimed in claim 10 wherein said plurality of teeth form an angle of about 60° with respect to each adjacent tooth.

12. A surgical forceps as claimed in claim 9 wherein one of said scissor arms has an outwardly curved end with a pawl formed on an outside surface of said curved end and the other scissor arm is provided with a ratchet assembly.

13. A surgical forceps as claimed in claim 12 wherein said ratchet assembly comprises:
a toothed bar which is adapted to engage said pawl to hold said forceps arms in a fixed position, said toothed bar being engaged and urged against said pawl by a spring member mounted on a scissor arm other than the scissors arm having a pawl.

14. A surgical forceps as claimed in claim 9 wherein said jaw member cutout notch area forms a window when the jaw members are closed.

15. A surgical forceps as claimed in claim 9 wherein said cylinder jaws extend from each respective arm at an angle of about 110°.

16. A surgical forceps comprising a pair of scissor arms rotatably connected together with the proximal ends forming hand grasping means and the distal ends provided with jaw members, each jaw member having a curved inner surface with a plurality of teeth and measurement markings placed at spaced intervals along an end surface of the first and second jaw members, each jaw member having a semi-circular body extending outward from each respective arm at an angle ranging from 90° to 130° so that when said scissor arms are closed together the jaw members ends move toward each other, each jaw member defining a cutout notch area allowing visual identification of a bone workpiece held therein; one of said scissor arms has an outwardly curved end with a pawl formed on an outside surface of said curved end and the other scissor arm is provided with a ratchet assembly with a toothed bar adapted to engage said pawl to hold said forceps arms and associated jaw members in a fixed position, said toothed bar being engaged and urged against said pawl by a spring member mounted on said other scissor arm.

17. A surgical forceps as claimed in claim 16 wherein said plurality of teeth comprises a plurality of spaced teeth positioned at the distal end of each jaw member running to the bottom of the cutout notch area.

18. A surgical forceps as claimed in claim 16 wherein said toothed bar is pivotally mounted in a yoke on said other scissor arm..

19. A surgical forceps as claimed in claim 16 wherein said spring member is a leaf spring and extends through said yoke.

20. A surgical forceps as claimed in claim 16 wherein said arms are rotatably connected together by pivot means.
